(19) United States
(12) Patent Application Publication
(10) Pub. No.: US 2026/0054053 A1
(43) Pub. Date: Feb. 26, 2026

(54) Series Multi-Blood Pump with Dual Activation for Pediatric Patients with Heart Failure

(71) Applicant: Drexel University, Philadelphia, PA (US)

(72) Inventors: Thomas Palazzolo, Philadelphia, PA (US); Amy L. Throckmorton, Cherry Hill, NJ (US)

(21) Appl. No.: 19/309,509

(22) Filed: Aug. 25, 2025

Related U.S. Application Data
(60) Provisional application No. 63/686,222, filed on Aug. 23, 2024.

(51) Int. Cl. A61M 60/802; A61M 60/122; A61M 60/232; A61M 60/237

(52) U.S. Cl. CPC A61M 60/802; A61M 60/122; A61M 60/232; A61M 60/237; A61M 2205/04; A61M 2205/3331; A61M 2206/20

(57) ABSTRACT

A mechanical circulatory support device is provided that includes a housing containing separate first and second pumps, each pump having an inlet, an outlet, and an impeller. The device also includes an actuation mechanism within the housing and is movable from a first position to a second position to divert blood flow within the housing to one of the pumps or to bypass blood flow relative to one of the pumps within the housing. In the first position, the actuation mechanism directs blood flow exiting the first pump into a bypass passage so that blood flow bypasses the second pump, and in the second position, the actuation mechanism directs blood flow exiting the first pump into the inlet of the second pump. The first pump is an axial flow pump and the second pump is a centrifugal pump, and the actuation mechanism includes flexible hosing.

### CROSS REFERENCE TO RELATED APPLICATIONS

This application claims the benefit under 35 USC § 119(e) of U.S. Provisional Patent Application No. 63/686,222, filed Aug. 23, 2024.

### BACKGROUND

The present invention relates to blood pumps, in particular, blood pumps for pediatric patients with heart failure.

Pediatric heart failure (HF) is a severe disease which requires intensive medical care and potential surgical intervention to save a child's life. Children can develop a wide variety of congenital and non-congenital disorders that can severely impair heart function and lead to HF; in the United States alone, about 40,000 children require immediate surgical intervention after birth due to congenital heart defects, and infants born with complex malformations to their heart chambers or surrounding vasculature may need multiple open-heart surgeries to build life-sustaining cardiovascular physiology.

Children may also develop acquired cardiac diseases, such as cardiomyopathy, at a young age; these acquired illnesses can arise from bacterial or viral infections and may lead to depressed ventricular function and eventual HF. Both acquired and congenital HF are associated with complications such as neurological impairment and growth delays.

Children exhibit significant differences in cardiac demands, anatomy, and blood rheology, along with other psychosocial and physiological differences that directly impact pediatric clinical care. Their cardiac output is far lower than adults; however, cardiac demand grows rapidly as children age, and patient anatomy changes in concert.

Accordingly, clinical teams face continual physiologic and anatomic challenges when treating pediatric acquired and congenital heart diseases, which in severe cases can progress to premature heart failure (HF).

The current standard of care for pediatric patients experiencing severe HF is heart transplantation; however, donor heart availability is a challenge in the pediatric population compounded by additional factors related to the patient pool, such as donor-patient size matching and the physical growth and development cycles that occur throughout childhood. Unlike in the adult population, there is a narrow window of suitable donors for a pediatric heart transplant due to growth cycles. Effective pediatric therapies, in contrast to the adult population, must adjust to patient growth; as cardiovascular demands increase, therapeutic treatments must respond and adjust to such demands in long-term care scenarios.

Thus, in pediatric HF, cardiac dysfunction can pose an imminent threat to a child's life, and heart transplant, if available, becomes the only lifesaving treatment option. While awaiting transplantation, patients may develop unstable cardiovascular physiology and may require mechanical circulatory support (MCS) from blood pumps such as a ventricular assist device (VAD). These assist the patient's heart by pumping blood in concert with the left ventricle (drives blood to the body) or the right ventricle (drives blood to the lungs). VAD technologies for adult HF patients readily exist; however, options for pediatric patients are limited due to patient size constraints, increasing cardiovascular demands of physical growth, and complications of physiological heterogeneity of anatomical defects and growth rates.

In situations where pediatric patients require long-term cardiovascular support to sustain life while awaiting a transplant, mechanical circulatory support (MCS) devices have become a vital treatment option. These devices provide mechanical assistance to a child's circulation, supplementing the pumping of the heart or replacing it outright. MCS devices have seen successful adoption in the adult population; analysis from the United Network for Organ Sharing (UNOS) correlates implementation of MCS technology with a 50% reduction in waitlist mortality. It is expected that MCS adoption rates will continue to rise in the future.

However, compared to the adult treatment paradigm, the current state of the art for pediatric MCS is lacking in several key areas. The first major gap in the standard of care is the number of available treatment options; only a small number of devices are currently FDA approved for usage in the pediatric population. The limited device pool has often forced the usage of adult devices in pediatric patients; this practice is known as “off-design” device usage. Distinct differences in pediatric biology, cardiac output, blood rheology, and support make adult devices poorly optimized for pediatric support; research shows that “off-design” usage can potentially lead to hemolysis, platelet activation, coagulation, thrombosis, and even neurological impairment or death. Thus, “off-design” operation should be limited whenever possible, but given the lack of available treatment options in pediatric care, it is often necessary.

The few devices which are FDA approved specifically for the pediatric population provide support in a narrow window of younger patients. However, their operating range is too limited to provide long-term support given the dynamic flow and pressure requirements of growing pediatric patients. For example, the Berlin Heart EXCOR device, an extracorporeal pediatric VAD, requires six different sizes to span the entire pediatric age range. As patients “size out” of their current devices, the patient must be either bridged to a new device, introducing significant potential for surgical complications, or the current device must be operated “off-design”.

The size of approved devices is also a factor; many of these devices are too large to allow for implantable support in younger children, which severely limits patient mobility and impacts quality of life. This compelling unmet clinical need gives rise to a strong research motivation in the scientific and medical communities towards the development of next-generation pediatric MCS technologies.

Adult devices have been used in children with some success, but operation of pump “off-design” is known to increase propensity for blood damage (hemolysis) and dangerous clotting (thrombosis). There is also no blood pump technology available that can support a pediatric patient from childhood through adolescence; there is an unmet clinical need to innovate safe and effective MCS for longer term usage across pediatric growth phases.

### SUMMARY

A mechanical circulatory support device is provided that includes a housing containing separate first and second pumps, each pump having an inlet, an outlet, and an impeller. The device also includes an actuation mechanism within the housing and is movable from a first position to a second position to divert blood flow within the housing to one of the pumps or to bypass blood flow relative to one of the pumps within the housing. In the first position, the actuation mechanism directs blood flow exiting the first pump into a bypass passage so that blood flow bypasses the second pump, and in the second position, the actuation mechanism directs blood flow exiting the first pump into the inlet of the second pump. The first pump is an axial flow pump and the second pump is a centrifugal pump, and the actuation mechanism includes flexible hosing.

### BRIEF DESCRIPTION OF THE DRAWINGS

FIG. 1A is a perspective view of a blood pump according to an embodiment.

FIG. 1B is a perspective view of an axial pump of the blood pump of FIG. 1A according to an embodiment.

FIG. 1C is a perspective view of a centrifugal pump of the blood pump of FIG. 1A according to an embodiment.

FIG. 1D is an exploded perspective view of the blood pump of FIG. 1A according to an embodiment.

FIG. 2A is a graph showing pressure generation capabilities of prior art devices.

FIG. 2B is a graph showing flow capacity capabilities of prior art devices.

FIG. 3A is a perspective view of a blood pump according to an embodiment.

FIG. 3B is a perspective view of the flow path through the blood pump of FIG. 13 according to an embodiment.

FIG. 4A is a perspective view of an activation mechanism according to an embodiment.

FIG. 4B is a perspective view of a blood pump having the activation mechanism of FIG. 4A according to an embodiment.

FIG. 5A is a perspective view of an activation mechanism according to an embodiment.

FIG. 5B is a perspective view of a blood pump having the activation mechanism of FIG. 5A according to an embodiment.

FIG. 6A is a perspective view of an activation mechanism according to an embodiment.

FIG. 6B is a perspective view of a blood pump having the activation mechanism of FIG. 6A according to an embodiment.

FIG. 7 is a workflow diagram according to an embodiment.

FIG. 8A is a perspective view of a flow path through the axial pump only according to an embodiment.

FIG. 8B is a perspective view of a flow path through the axial pump only according to an embodiment.

FIG. 8C is a perspective view of a flow path through the axial and centrifugal pumps according to an embodiment.

FIG. 8D is a perspective view of a flow path through the axial and centrifugal pumps according to an embodiment.

FIG. 9A is a chart of k-epsilon and SST simulation results at multiple pump speeds for an axial pump flow path only according to an embodiment.

FIG. 9B is a chart of k-epsilon and SST simulation results at multiple pump speeds for a double pump flow path according to an embodiment.

FIG. 10A is a perspective view of an activation mechanism according to an embodiment.

FIG. 10B is a perspective view of an activation mechanism according to an embodiment.

FIG. 11A is a perspective view of an activation mechanism according to an embodiment.

FIG. 11B is an experimental setup for testing the activation mechanism of FIG. 11A according to an embodiment.

FIG. 11C is a perspective view of the activation mechanism of FIG. 11A in a first position according to an embodiment.

FIG. 11D is a perspective view of the activation mechanism of FIG. 11A toggled to a second position according to an embodiment.

FIG. 12 is a perspective view of a curved activation mechanism according to an embodiment.

FIG. 13 is an experimental setup according to an embodiment.

FIG. 14A is a chart of experimental results and k-epsilon and SST simulation results at multiple pump speeds for an axial pump flow path only according to an embodiment.

FIG. 14B is a chart of experimental results and k-epsilon and SST simulation results at multiple pump speeds for a double pump flow path according to an embodiment.

FIG. 15A is a perspective view of an activation mechanism according to an embodiment.

FIG. 15B is perspective view of an activation mechanism according to an embodiment.

FIG. 15C is a perspective view of a front of an activation mechanism according to an embodiment.

FIG. 15D is a perspective view of a rear of the activation mechanism of FIG. 15C according to an embodiment.

### DETAILED DESCRIPTION

For simplicity and illustrative purposes, principles of embodiments are described below by referring primarily to examples thereof. In the following description, numerous specific details are set forth to provide a thorough understanding of the embodiments. It will be apparent to one of ordinary skill in the art that the embodiments may be practiced without limitation to these specific details. In some instances, well known methods and structures have not been described in detail so as not to unnecessarily obscure the embodiments.

According to embodiments disclosed herein, a medical mechanical circulatory support device is provided that includes multiple pumps. For example, an axial pump may be used for initial circulatory assistance in younger patients; then, an internal activation mechanism triggers a centrifugal pump to activate in line with the axial pump, thereby providing additional pressure and flow to match pediatric patient growth cycles.

The unmet clinical need for this type of device is supported by a systematic literature review of the competitive landscape of pediatric MCS devices. FIGS. 2A and 2B show design requirement achievement for pediatric blood pumps: Pressure generation (FIG. 2A) and flow capacity (FIG. 2B). As shown in FIGS. 2A and 2B (i.e., a snapshot of pump pressure/flow performance data for pediatric VADs), no single blood pump was able to produce sufficient flow capacity to cover the pediatric age range. While the top eleven devices listed in FIG. 2A meet design target requirements concerning systemic pressure rise (mmHg), none of devices also meet design target requirements concerning blood flow (L/min) as shown in FIG. 2B. Many of the devices also failed to meet additional design requirements related to blood damage, device size, and more. This literature review is evidence of limitations that necessitate the development of a more versatile blood pump technology for pediatric patients with HF for use as a bridge therapy, as a long-term chronic care solution, and as a viable treatment option to span the pediatric age range, from childhood through adolescence.

According to embodiments disclosed herein, a medical device technology is provided that: A) integrates multiple blood pumps in a single device housing; and B) facilitates strategic activation of these pumps in different conditions to boost pressure and flow capacity during growth and development. Such a device improves the current standard of care by providing effective MCS across the pediatric age range. Merely for purposes of example, devices having multiple pumps are disclosed in U.S. Pat. Nos. 9,919,085, 10,814,053, 11,998,728 and 12,239,831.

According to embodiments disclosed herein, a pediatric ventricular assist device is disclosed that includes a multi-blood pump design, incorporating an axial and centrifugal pump in a single device. For younger pediatric patients, the smaller axial blood pump is used to provide appropriate blood flow and pressure levels. When capacity and pressure requirements increase due to patient growth, the centrifugal pump operates together with the axial pump in series; the centrifugal pump provides a further pressure and capacity boost for older or larger pediatric patients. Compact axial impellers are suited to low-flow conditions and allow for greater design variation within size constraints. However, at higher flow rates, an axial-flow impeller would require much higher rotational speeds to generate sufficient pressure; this would risk increasing fluid stress levels and potentially contribute to hemolysis. Operating the axial pump in a tandem series configuration with a centrifugal pump (i.e., larger impeller surface area, thus higher pressure at lower RPM), provides sufficient overall pressure boost at capacity, while mitigating irregular flow patterns and fluid stresses. In this tandem configuration, blood flow first moves through the axial pump for an initial pressure boost and then through the centrifugal pump for a secondary pressure boost in support of the child's growth demands.

An internal activation mechanism facilitates operational switching from single to dual-pump support; this is accomplished by a blood flow directional shift from the outflow of the axial pump into the centrifugal blood pump (dual series, tandem operation). A magnetic suspension motor drive system for both impellers can be utilized; the use of magnetic levitation enables wider clearances between stationery and rotational surfaces, lowering fluid stresses and risk of blood damage. The compact device is designed for implantable support in patients with a body surface area (BSA) as low as 0.7 m² (a 50th percentile 4 year old male) while providing sufficient support up to 5 L/min. Extracorporeal support is an option for smaller patients.

Hydraulic performance goals are 1-3 L/min with a minimum pressure rise of 50 mm Hg for axial-only operation, and an increase in pressure and flow capacity to 120 mm Hg and 3-5 L/min for combined dual-support, which will allow for robust single device support across the pediatric age range. These performance targets were derived from studies detailing pediatric cardiac requirements, published literature, and consultation with clinical experts.

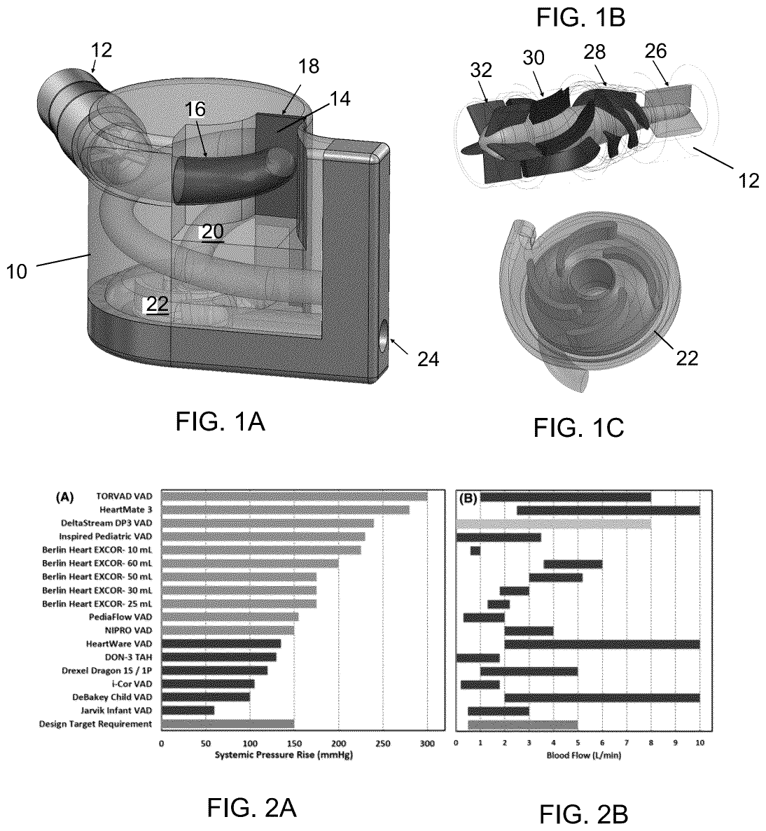

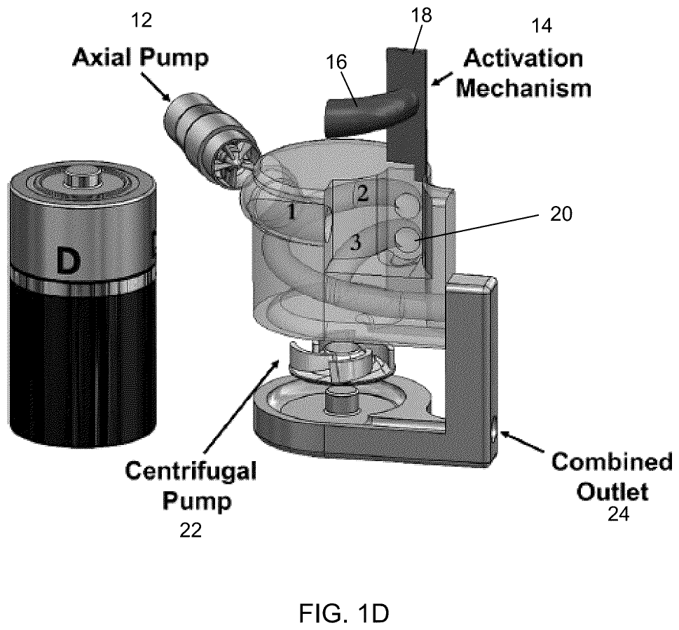

According to an embodiment shown in FIGS. 1A-1D, a device 10 has two moving parts during normal operation: an axial impeller; and a centrifugal impeller. As best shown in FIG. 1A, the device 10 includes an axial pump 12, an activation mechanism 14 including a flexible-tube portion 16 and a moving portion 18, a centrifugal pump inlet 20 a centrifugal pump 22, and a joint device outlet 24. The activation mechanism 14 permits the axial pump 12 and centrifugal pump 22 to be arranged in series.

The axial pump 12 is shown in greater detail in FIG. 1B. The housing of the axial pump 12 houses inducer blades 26, a rotating impeller section 28, stationary diffuser blades 30, and flow straightener blades 32. The centrifugal pump 22 is shown in FIG. 1C. An exploded view of the device is shown in FIG. 1D. A flow path output from the axial pump 12 is shown as path “1” in FIG. 1D. This path leads into the activation mechanism 14. If the activation mechanism is aligned to direct the flow into bypass flow path “2”, then the flow bypasses the centrifugal pump 22 and is directed to the outlet 24. Alternatively, if the activation mechanism is aligned to direct the flow into the flow path “3”, the flow enters the inlet of the centrifugal pump 22 before being directed to the outlet 24. For purposes of demonstrating the size of the device 10, a D-size battery is illustrated therewith in FIG. 1D for size comparison.

Magnetic bearings levitate the impellers, which: 1) allows for wider clearances between rotating and stationary surfaces; 2) reduces component wear to increase device lifespan; and 3) lowers fluid stresses, mitigating the risk of thrombosis and hemolysis. The device 10 of FIGS. 1A-1C does not use mechanical or biologic valves, further lowering thrombosis risk. The device 10 is compact and delivers physiologic pressures and flows for pediatric patients with varying levels of heart failure and growth demands.

The integration of an activation mechanism 14 to govern the operational coupling of two blood pumps from single-pump support to dual-pump support provides a design attribute unlike available VAD technology. This enables the device 10 to provide longer-term MCS for pediatric patients over growth and development phases, improving upon current design shortcomings and addressing a significant clinical need.

Strong data in the design of a hybrid, dual-support pediatric VAD is provided by embodiments disclosed herein. Axial and centrifugal pumps have been developed through modeling and testing; the devices have achieved target pressure and capacity requirements. The innovative activation mechanism that governs operational control of the pumps is integrated into the device. Accordingly, a novel, dual-support VAD configuration and activation mechanism as a therapeutic option for long-term MCS of pediatric patients with HF are provided.

By way of example and not by way of limitation, the specific aims of the embodiments disclosed herein include the following:

Aim 1: Establish a novel activation mechanism and VAD configuration that achieves design requirements by iterative modeling. The device housing and blood flow paths must meet design requirements of compact size, switching between operating modes (axial-only and combined dual-pump support), sufficient capacity and pressure generation, motor/magnetic suspension accommodation, and minimization of irregular blood flows that may trigger blood damage or thrombosis. Multiple designs have been investigated using computational analysis, for both flow domains, under steady and transient blood flow conditions.

Aim 2: Prototypic designs of the activation mechanism to achieve target motility and sealing requirements has been investigated. These are evaluated through in vitro hydraulic studies testing repeatable, failsafe activation mechanism operation, mitigation of deleterious blood flow conditions, controller testing and troubleshooting verification, and assessment of blood damage metrics such as plasma free hemoglobin for a range of operational blood flow conditions.

Aim 3: Characterize the ability of the coupled activation mechanism and dual-support blood pumps to achieve design requirements through in vitro testing. The goal is to experimentally demonstrate the ability of the device housing, pumps, and flow paths to provide adequate pediatric support. This can be validated with computational results through in vitro testing of prototypes against design requirements of size, pressure-capacity generation, and activation goals of leak-free operation and transition from single-pump to dual-pump support.

Accordingly, the embodiments disclosed herein address an unmet clinical need and provide a double-blood pump pediatric VAD technology that serves as a single-device solution for patients requiring MCS support across the pediatric age range.

These embodiments offer several distinct and advantageous design attributes:

1. Multi-blood pump design which uniquely incorporates both an axial and centrifugal impeller within a single pump housing. This allows for multiple modes of operation: axial blood pump only, or axial and centrifugal blood pumps operating in tandem.

2. Novel device activation mechanism that facilitates the reliable activation of a secondary flow path to boost capacity and pressure generation during patient growth and development.

3. Minimal moving parts through the incorporation of magnetic suspension for both the axial and centrifugal pumps, allowing for wider clearances between stationery and rotating surfaces of the pumps and reducing blood shear stresses, lowering the risk of blood cell damage leading to hemolysis and thrombosis.

4. Compact design, implantable in patients with a body surface area (BSA) as low as 0.7 m² (a 50th percentile 4-year-old male), and extracorporeal support for smaller patients.

The pediatric dual-support VAD utilizes magnetic bearing technology to levitate the impellers, thus enabling a longer operational lifespan (15-20 years) due to lower wear and wider clearances between the rotating impellers and pump housings (5 times wider than mechanical bearings) that reduce fluid shear stresses and lower the potential for hemolysis and thrombogenicity. The design avoids the use of constantly cycling mechanical or biologic valves that may fail prematurely due to repetitive opening and closing.

Incorporation of the activation mechanism allows for two distinct operational modes-single pump (axial only) operation and double-pump combined operation. The operational mode can be selected to specifically tailor to the current support needs of the patient.

In the low flow, axial-only configuration, the axial blood pump is used by itself to provide support to younger pediatric patients. As capacity and pressure demands increase in line with growth and development, the activation mechanism can be triggered when needed and facilitate a blood flow shift from the outflow of the axial pump into the centrifugal blood pump for additional pressure and capacity. In older pediatric patients, both pumps will be employed in series to meet physiological needs for the patient.

The axial blood pump of some of the embodiments disclosed herein is designed to generate flows from 1-3 L/min with a maximum pressure rise of 50 mmHg, while the combined double-pump support mode is designed to produce up to 120 mmHg of pressure from 3-5 L/min. Accordingly, the devices disclosed herein are designed to support pediatric patients with HF.

The medical technology of the embodiments integrates multiple blood pumps that facilitate the activation of a secondary pump in line with an initial support pump to boost pressure and flow capacity for growth and development.

According to embodiments, the device configuration and activation mechanism incorporate both axial and centrifugal pumps into a single compact device housing and allows for effective switching between operational modes to provide robust long-term support across the pediatric population. The overall device design must meet device size and fitment constraints, creation of an activation mechanism that meets design requirements, and iterative refinement of the device and flow path design through computational fluid dynamics (CFD) analysis. Multiple turbulence models have been utilized to ensure alignment with in vitro experimental data. The double-blood pump pediatric VAD device is able to provide effective MCS for growing pediatric patients over long support durations, while mitigating device footprint.

According to an embodiment, the device body and flow paths, were drafted using Computer-aided design (CAD), were iteratively improved through Computational fluid dynamics (CFD) modeling, which numerically approximates a solution to the Navier-Stokes equations of fluid motion through iterative calculation. This allows for accurate simulation of device performance and flow path characteristics in silico. Within the computational space, a variety of turbulence models which simulate the characteristics of turbulent fluid flow was utilized. Utilizing the Kepsilon turbulence model, CFD simulations of pump designs demonstrated that the pumps met all design criteria, outside of blood shear stress values at high pump rotational speeds.

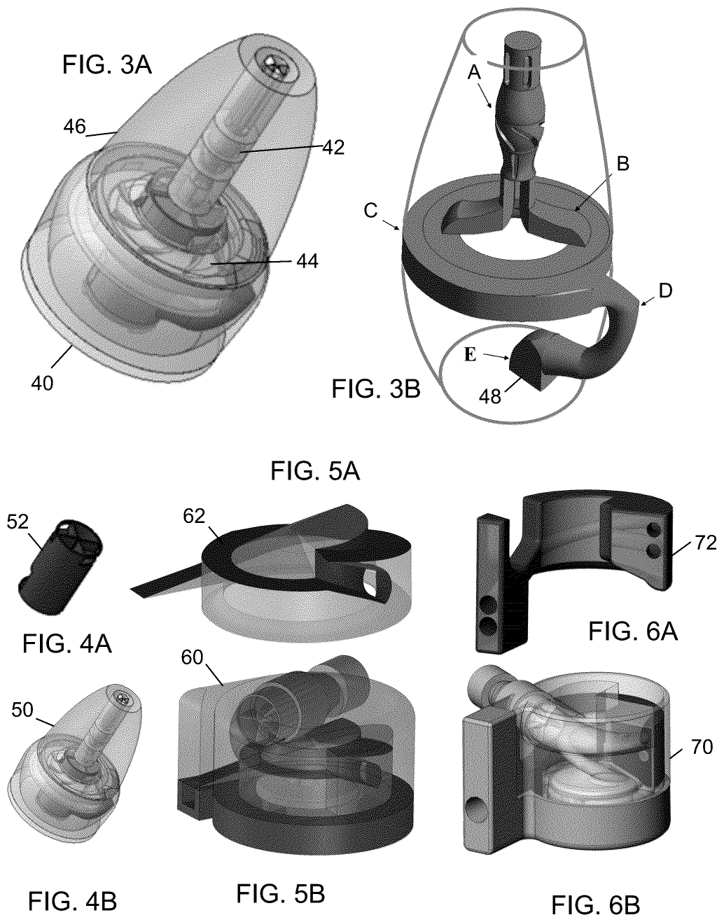

The pump housing and activation mechanism of one such embodiment is shown in FIGS. 3A and 3B in which the axial pump and centrifugal pump are in line (i.e., in series). FIG. 3A shows a device 40 having an axial pump 42 and centrifugal pump 44 contained within a housing 46. FIG. 3B shows the blood flow paths inside of the device housing 46. The path includes a path A through the axial pump 42, split paths B entering into the centrifugal pump volute C, path D from the outlet of the centrifugal pump 44, and path E provided as a 90° bend to align with a main outlet 48 of the device 40.

The inline orientation results in an overall device length of about 90 mm, which may be too long to allow for implantable support in some younger pediatric patients. The combined flow path outlet rejoins the main outlet through a 90° bend, and the axial flow path cross-sectional area rapidly decreases by 50% as flow passes through the device 40. These constraints may impact the performance of the device in some situations. Accordingly, a VAD configuration and activation mechanism which meet pump performance requirements while also meeting qualitative and quantitative device requirements related to device size, alignment, and pressure/flow may be desired.

The goal of determining the geometry and action of the activation mechanism is a key element of the device design which allows for both single-pump and combined, tandem operation. An effective activation mechanism design should be compact in size, exhibit effective switching between operating modes (axial-only and axial+centrifugal in series), exhibit optimal flow through the impellers and blood flow paths, and minimize irregular blood flows that could give rise to blood cell trauma or flow stasis.

Given some of the limitations of the above referenced design, other embodiments of an activation mechanism were assessed against quantitative and qualitative design requirements. Utilizing CAD software, several embodiments for a new activation mechanism were contemplated.

One contemplated embodiment is shown in FIGS. 4A-4B. A device 50 shown in FIG. 4B incorporates two fully separate flow paths for the axial-only and the combined series configuration. The activation mechanism 52 shown in FIG. 4A rotates 90 degrees along its axis to activate a secondary flow path. This design requires an automatic valve or pivoting component at the initial flow branch to direct flow and seal off the unused flow path, and a passive valve at the outlet flow junction to prevent backflow. Thus, this design requires at least two moving parts.

Another contemplated embodiment is shown in FIGS. 5A-5B. A device 60 shown in FIG. 5B incorporates two fully separate flow paths for the axial-only and the combined series configuration. The activation mechanism 62 shown in FIG. 5A includes a shroud with a flow path square in cross-section and centrifugal impeller walls integrated into the mechanism.

The shroud-type mechanism 62 utilizes the mechanism itself as an integral component of each flow path and saves space by integrating the mechanism into the outer walls of the centrifugal impeller. Thus, device 60 may be constructed sufficient compact for implantation in patients with body surface areas as small as 0.7 m². The shroud-type activation mechanism 62 utilizes a rectangular flow path in the mechanism to reduce device space.

A further contemplated embodiment considered integration of the axial and centrifugal blood pumps into the device housing instead of mounting the axial impeller in line with the centrifugal impeller, which significantly impacts the length of the device. Thus, the axial pump was placed parallel to the centrifugal pump, separate from the main pump body. This permitted reduced device height but increased device length beyond the overall device size requirements. Further investigation of pump placement concluded by mounting much of the axial impeller within the main body of the device, and constraining separation of the two flow path domains within the activation mechanism itself.

Accordingly, the culmination of activation mechanism design and pump packaging against device size requirements, is illustrated by the embodiment in FIGS. 6A-6B. A device 70 shown in FIG. 6B incorporates two fully separate flow paths for the axial-only and the combined series configuration. The activation mechanism 72 shown in FIG. 6A includes flow paths for both the axial-only and combined operation modes and travels through the mechanism itself. The flow domain maintains paths having a circular-cross section and the activation mechanism is independent from the centrifugal pump domain. These changes improve the flow profile, lower blood damage, and enhance sealing, while also allowing for independent improvement of the impeller domain. This design also met size constraints.

The performance of the device compared against design criteria derived from literature and device geometric constraints was investigated. This was accomplished through CFD analysis, utilizing a prioritized design improvement process to modify key design characteristics such as the flow path diameter, centrifugal inlet location, flow path diameter change, and flow path curvature. These parameters were ordered through evaluation of parameters such as the minimum flow path diameter have an outsized effect on pump performance while other parameters such as flow path curvature have less of an impact. Design optimization was accomplished with modification of key parameters.

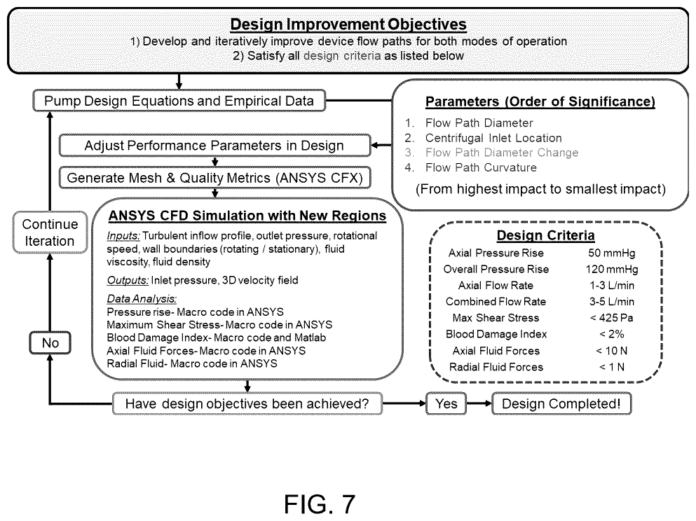

FIG. 7 illustrates a flow chart of the iterative design improvement process. Pump parameters are modified in order of significance (highest impact on pump performance from design changes to lowest impact on pump performance). The main design improvement objective was to ensure that the blood flow path design did not impact the ability of the pump designs to meet their previously-met performance requirements. These design requirements are as follows: 1-3 L/min of flow in the axial-only flow path, 3-5 L/min of flow in the combined flow path, axial pump speeds of 10,000-13,000 rpm, and centrifugal pump speeds of 5,000-6,000 RPM, 50 (axial)/120 (combined) mmHg maximum pressure rise.

Simulations were used to calculate 1) the pressure generated by the impeller(s) at a range of flow rates, 2) the overall device pressure rise for axial and combined flow paths, 3) fluid velocity streamlines throughout the flow paths, which were used to investigate areas of vorticity or flow stagnation, and 4) the predicted level of blood damage across the device utilizing a power law relationship.

In accordance with standard practice in the field, ANSYS CFX was employed for computational modeling of blood flow through the pump designs. Mesh size and quality are known to impact the accuracy of the solution, so the following mesh quality metrics were employed for modeling to ensure simulation accuracy: aspect ratio (a measure of mesh element deviation from equal-length size, maximum value of 100); skewness (the difference between cell shape and the shape of an equal-volume equilateral cell, maximum value of 0.95, average value below 0.33); and element quality (an ANSYS-specific value which measures the regularity of mesh element shapes, average value of at least 0.5). These metrics were used to ensure that the model would produce results with high predictive value. It is also critical to ensure that the mesh is dense enough that simulation results are no longer affected by increases in mesh density; this was accomplished through a grid independence study, an evaluation which compares solution results at different mesh resolutions to determine acceptable mesh fidelity through quantifying a percent difference in key parameters (less than 3% variance in simulated pump velocities and pressures at key locations).

Flow conditions in the pumps involve non-laminar, turbulent flow conditions. Reynolds number estimations were greater than 4,000 in the pump impeller domain and are expected to be much higher at the blade-tip clearances; thus, the CFD simulation incorporated a turbulence model. Two turbulence models were chosen, in order to provide a comparative basis between each model and contribute to the body of research with full double-blood pump flow path simulations. The first was the k-epsilon model; k-epsilon robustly models bulk fluid flow but, unlike more complex models, does not simulate boundary layer behavior and flow separation. In contrast, the Menter Shear Stress Transport (or SST) turbulence model leverages the aforementioned k-epsilon model in the bulk flow region while also incorporating the k-omega model, which does capture viscous boundary layer behavior, at the edges. SST toggles between these two turbulent flow modes using a blending function to determine which model to apply at each node. Utilization of both models elucidate the effect of boundary layer separation/edge effects on device performance.

The accurate modeling of blood behavior is another consideration; blood is a shear-thinning non-Newtonian fluid (at shear rates below 100 s⁻¹, shear-thinning effects are minimal). Pediatric patients exhibit variation in blood rheology with hematocrit values ranging from 20% to 60%; thus, a non-Newtonian shear thinning blood model was utilized to more robustly model blood behavior.

To first validate the simulation design, the pump designs themselves were evaluated in simulation with k-epsilon studies. With this condition satisfied, evaluation of the full device flow paths, utilizing both turbulence models discussed above, were accomplished. The designs were refined using the prioritized device design improvement process shown in FIG. 7. Changes were made until a satisfactory design was generated that could meet all design requirements. Geometric adjustments included an increase in the minimum flow path diameter from 4.5 mm to 7 mm, relocation of the centrifugal pump inlet from 8.15 mm off centerline to 5 mm off centerline, and complete elimination of diameter change in the outlet flow path.

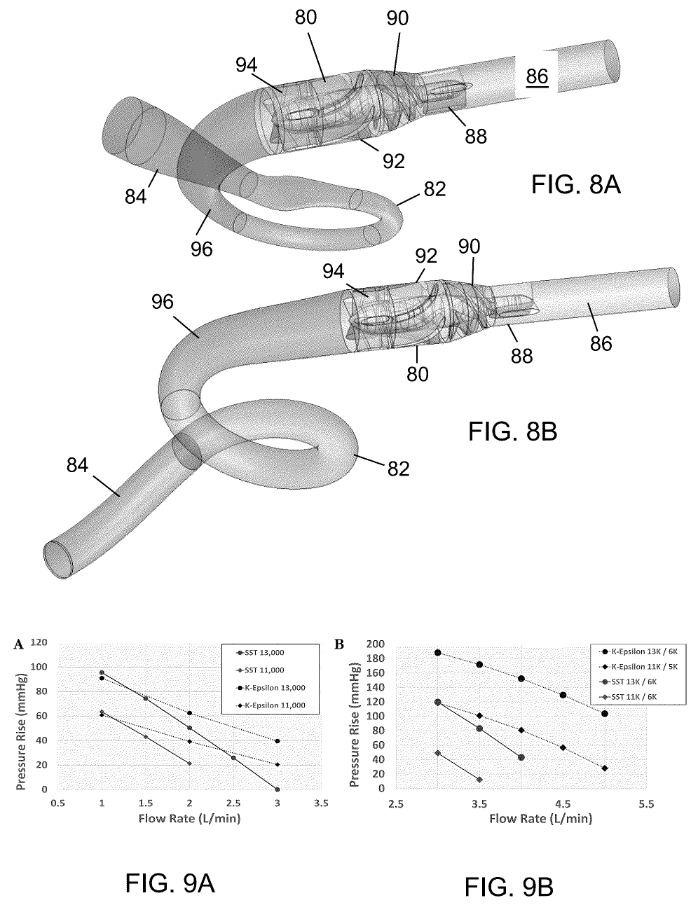

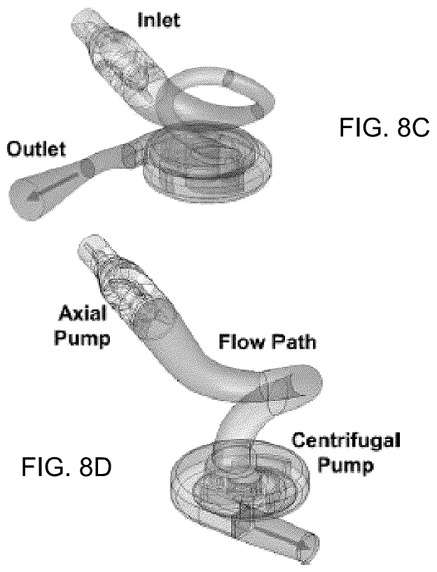

FIGS. 8A and 8B provide a comparison of flow paths through the axial pump only. In FIG. 8B, the flow path has more gradual diameter reduction after the axial pump 80, larger flow path diameter through the outlet volute 82, and consistent outlet diameter 84 as compared to the flow path in FIG. 8A. Section 86 of the flow path is the device inlet leading into the static inducer 88 of the axial pump 80. Section 90 of the flow path is the Impeller region where the blades of the axial pump rotate to generate pressure, section 92 of the flow path includes static diffuser and straightener regions 94 of the axial pump, and section 96 is the outflow region.

FIGS. 8C and 8D provide a comparison of flow paths through the axial pump and the centrifugal pump.

FIGS. 9A and 9B illustrate the hydraulic performance data from the design, showcasing both the k-epsilon and the SST model results. Comparison of k-epsilon and SST simulation results at multiple pump speeds for the axial-only flow path (FIG. 9A) and the combined, double-pump flow path (FIG. 9B).

Accordingly, embodiments met device performance design requirements with the k-epsilon model; both the axial-only and combined flow paths generated sufficient pressure and flow to provide single-device support across the pediatric age range. However, a marked discrepancy was noted between k-epsilon model results and SST model results. In both flow paths, the SST model exhibited a steeper decline in pressure generation compared to k-epsilon, especially in the combined flow path, where flow is higher. This trend was consistent across all rotational speeds. In vitro experimental test data allowed for direct comparison against, and verification of, computational results.

Accordingly, a design which meets pump performance goals for pressure and flow generation based on steady-state simulation results in the k-epsilon simulation domain. To provide further insight into device behavior, transient simulations have been performed to capture time-dependent blood flow features.

The dynamic parts of the device are not actually moving; domain motion is accounted for relative to stationary domains through the incorporation of momentum terms to nodes in the rotating domain. This provides a time-averaged solution, but only investigates a single impeller position divorced from potential time-dependent effects.

Three types of time-dependent simulations have been performed to capture transient effects: 1) quasi-steady state; 2) translating rotating sliding interface (TRSI); and 3) time-varying boundary conditions (TVBC).

Quasi-steady state simulations provide a series of steady-state flow snapshots that develop from the fluid's previous state of motion. At the operational design points of the axial and centrifugal pump, a quasi-steady simulation was conducted for each pump independently, and then a combined, dual-pump (i.e., axial+centrifugal) quasi-steady simulation was performed at a near design point (i.e., physiologically realistic performance). These are completed at 5° increments (i.e., 72 simulations to achieve a full 360 degree rotation).

TRSI simulations investigate impeller blade rotation as a function of time, building a solution from each previous time step. This incorporates the history of the fluid physics, and thus, the effect of time dependent transient effects. To fully resolve transient effects, TRSI are run for three full rotations (i.e., 1080°) of the impellers. At the operational design points of the quasi-steady study, TRSI simulations are conducted for each pump separately, and then a combined, dual-pump TRSI model is executed. Two operational rotational speeds were considered. The iterative time-step is determined according to Nyquist frequency requirements and blade passage frequency for each pump.

TVBC simulations incorporate the pulsatile nature of the human circulatory system. This is accomplished by defining a pulsatile waveform at the inflow of the axial flow blood pump. This is derived from published pediatric cardiac cycle data and then scaled to meet the flow demands and heart rates under investigation. TVBC simulations are run for three full cardiac cycles at 75 and 90 beats per minute. TVBC simulations are conducted for the axial pump only and then the combined, dual-tandem TRSI model is executed. Two operational rotational speeds were considered.

Fully Combined Transient Simulations involve the combined, integrative modeling of both TRSI and TVBC effects. This simulation case is only used for the combined operational mode. The iterative time-step is determined according to Nyquist frequency requirements and blade passage frequency for each pump. Operating conditions in accordance with prior modeling of TRSI and TVBC settings were used for comparison purposes.

While the simulations performed mimic the magnetically-suspended pump designs, the use of shaft drive for experimental testing necessitates changes in geometry to incorporate drive shafts, bearings, and additional elements. This could introduce a discrepancy in simulation results when compared to in vitro testing. Thus, a fluid dynamic model of the shaft-drive axial pump setup in CFD, using the SST turbulence model and maximum pump speeds is used. For direct comparison, operational conditions are simulated in line with the magnetically-levitated models.

The primary outcome produced a complete device design configuration that meets qualitative and quantitative device design objectives to allow for effective support across the pediatric age range. A device that meets design requirements in the k-epsilon simulation domain, and also meets constraints related to device size and ability to provide implantable support has been developed.

Prototypic designs of the activation mechanism that achieve target motility and sealing requirements has been developed. The objective is to develop and test a prototypic design for the novel activation mechanism which demonstrates repeatable, failsafe operation with minimal modification to device size/flow path and limited introduction of deleterious blood flow conditions. The approach involved development of activation mechanism concepts and testing of developed designs in vitro to verify expected performance. The rationale is that the resultant activation mechanism design will allow for safe and effective transition between device operating modes, allowing for longer-term support that adjusts to patient growth and development cycles.

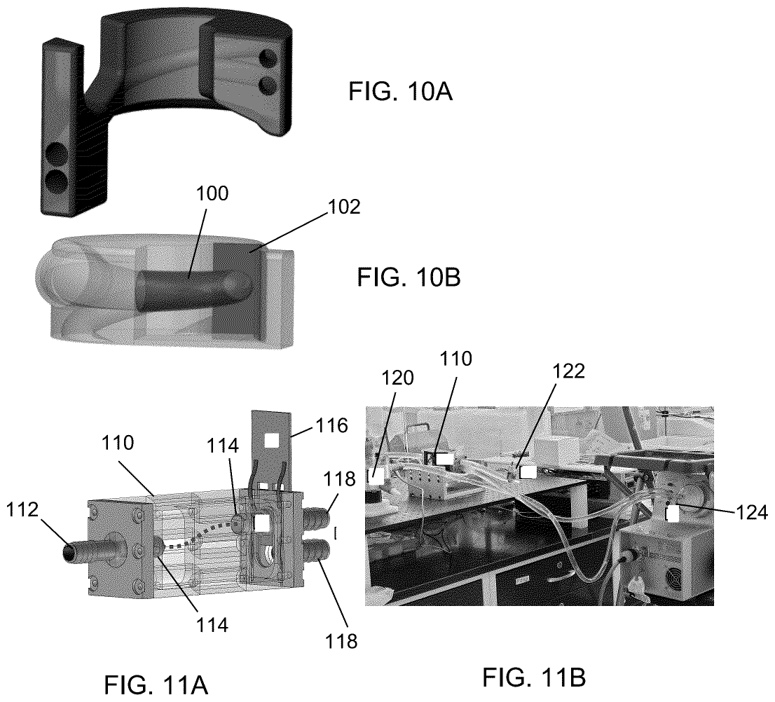

There is no device on the market today which utilizes two blood pumps and multiple operating modes to provide long-term pediatric MCS. Activation mechanisms according to embodiments that make multi-pump support possible in a single device, thus widening the operating range, are shown in FIGS. 10A and 10B. The activation mechanism in FIG. 10A is the mechanism previously discussed with respect to FIG. 6A. The activation mechanism in FIG. 10B is the mechanism previously discussed with respect to FIG. 1A (i.e., flexible-tubing design). The activation mechanisms consist of a single piece, incorporate the blood flow path itself within the mechanism to ensure that the device would not breach size constraints, and mitigates potential for hemolysis or thrombosis.

The ability of the devices to meet these requirements was evaluated. Such evaluation revealed several considerations with respect to activation mechanism design. The first was the method of action; while the activation mechanism itself may consist of a single part, the incorporation of the activation mechanism into the flow path requires multiple seals against moving surfaces and precise positioning to ensure minimal impact on blood flow. As many as four separate seals may be needed through the combined double-pump flow path. The size of the activation mechanism itself also presents potential difficulties with incorporation of magnetic suspension components.

Accordingly, instead of two completely separate flow paths contained within the activation mechanism as shown in FIG. 10A, a design as shown in FIG. 10B that utilizes flexible tubing 100 that moves to switch between two static flow paths. A thin, independently sealed flat plate 102 at the junction of the flow paths is the only moving part of this design. This minimizes space, number of seals, and size of moving parts; it also allows large portions of the device flow path, formerly housed within a large moving component, to be incorporated into the fixed main pump body.

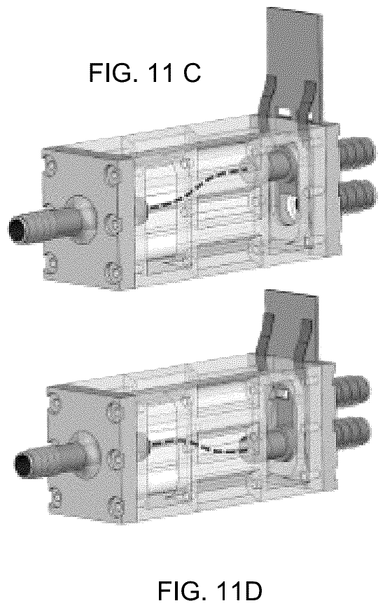

As a proof of concept, a simplified prototype design (see FIGS. 11A and 11B) was created and tested in vitro to evaluate the potential impact of the design on flow/pressure generation. The goal of this study was to ensure that the isolated design could withstand maximum physiological pressures and flows while retaining a robust seal.

An activation mechanism 110 is shown in FIG. 11A. The activation mechanism 110 includes a joint device inlet 112, barbed hose connectors 114 for flexible tubing, a moving plate portion 116, and multiple device outlets 118, which can be toggled through movement of the plate 116. The plate 116 is independently sealed and translates the flexible flow path between operational modes, effectively toggling between axial-only and combined operation while halving the number of seals required for the combined flow path. FIGS. 11C and 11D show the flexible tubing or hose toggled between being aligned with the upper outlet and lower outlet, respectively.

An illustration of the experimental setup is provided in FIG. 11B. The setup includes a pressure vessel tank 120, the activation mechanism 110, a flow restriction clamp 122 on the secondary outlet, and a BioMedicus ⅜″ FloPump 124. Thus, open portions of the device flow path (such as the secondary outlet) were sealed off to create a closed system. Utilizing the BioMedicus ⅜″ FloPump centrifugal blood pump (International BioPhysics Corp, Austin, TX), the device was tested using water at a maximum flow rate of 7 L/min for 30 minutes. At that point, a flow restriction was introduced into the pump circuit to restrict flow to 3 L/min (the “operational switchover” flow rate) and generated a pressure rise of 150 mmHg. Both tests ran above the maximum pressure rise and flow rate of the device (5 L/min and 120 mmHg) to introduce a factor of safety. During the test, leaks appeared at the connection between the hose and the flexible tubing; however, evaluation of the device both during and after the test revealed that this leak occurred due to mechanical failure of the hose connector. No leakage was observed in the chamber of the activation mechanism itself, and the internal seal was found to be dry when disassembled.

Thus, the initial design prototype demonstrated robust performance and sealing.

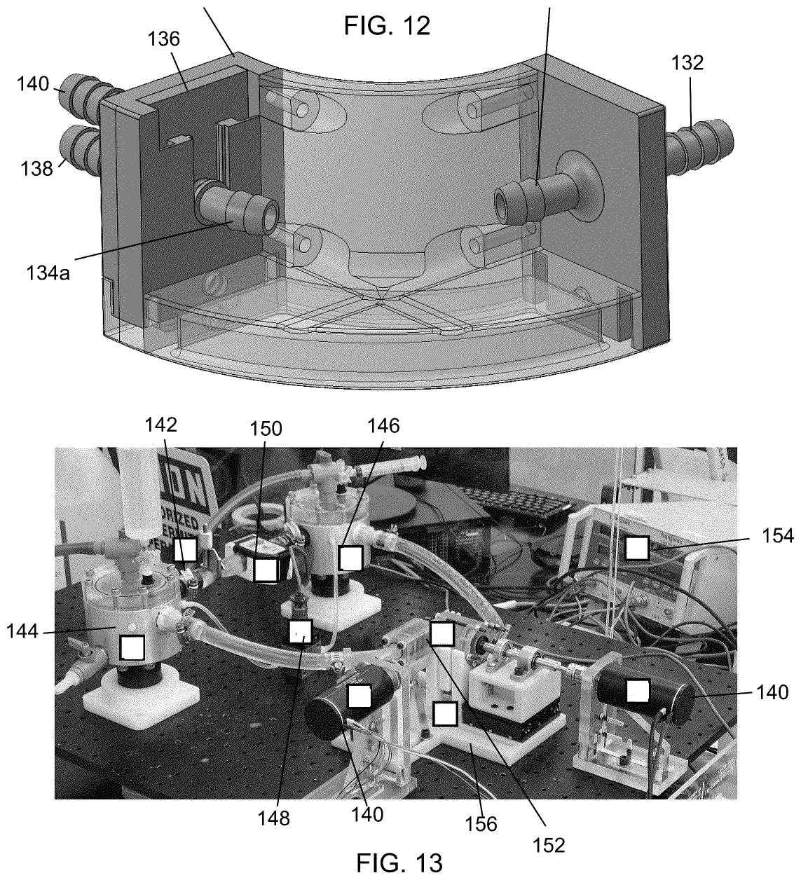

An additional embodiment of an activation mechanism 130 is shown in FIG. 12. The activation mechanism 130 is curved and similar to activation mechanism 110 includes an inlet 132, barbed connectors 134 to connect to flexible hosing (not shown), a movable plate 136 on which the barbed connector 134a is connected, and a pair of outlets, 138 and 140, to which the barbed connector 134a can be aligned with via movement of the plate 136.

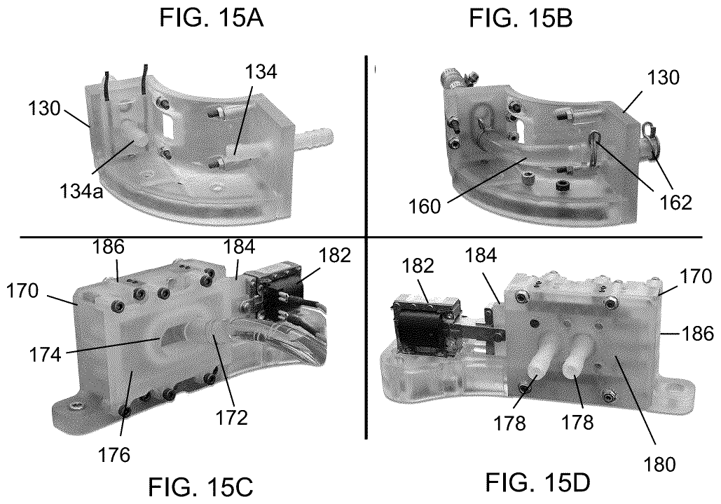

The activation mechanism 130 is also shown in FIG. 15A and is shown in FIG. 15B with a single flexible hose 160 secured to the barb connectors, 134 and 134a, with wire springs 162. Neodymium magnets were added to increase the compressive sealing force between the plate and the outlet body. The magnets also passively improve alignment between the plate and housing. The mechanism in FIG. 15A includes a plate compressed against a rear housing wall with a pair of springs. The mechanism in FIG. 15B includes a flat back plate, pressed against the rear wall and outlets with a compressible seal. The seal provides consistent tunable pressure to the back of the outlet. Durometer 70D Buna-N square O-rings or Durometer 55D round FEP-encapsulated O-rings were used to reduce distortion of the seal in situ and decrease friction between the plate and the housing.

An additional embodiment of an activation mechanism 170 is shown in FIGS. 15C and 15D. The inlet 172 extends through a slot 174 formed in a wall 176 of the activation mechanism 170. The inlet 172 can be slid in the slot to align the inlet to either of the two outlets 178 in a rear wall 180 of the activation mechanism 170. Movement of the inlet 172 within the slot 174 can be accomplished with magnets.

By way of example, a linear actuator 182 is able to move a plate 184 extending within housing 186. The plate 184 can be moved in situ without manual intervention. In the activation mechanism shown in FIGS. 15C and 15D, the plate slides from side to side via an opening in an end wall of the housing 186. This ensures that the actuator 182 is not affected by gravity. A 120 V actuator with a maximum pulling force of 155 oz. (McMaster-Carr, Elmhurst, IL, US) was utilized and was able to pull the plate toward the main actuator body to align the inlet 172 with one of the outlets 178 in the rear wall 180. Pulling force was modulated through a 1000-Ω potentiometer (Ohmite RGS1K0E, Ohmite, Warrenville, IL, US), which allowed for precise control of power delivery. The actuator was a pull-only design; thus, three linear springs were used to return the plate to the primary outlet once the actuator was powered off.

The free-floating plate utilized in actuator mechanism 130 can be mounted on two sets of guide rails, one on each edge of the plate. The rails reduce friction and fix the horizontal positioning of the plate, passively compressing the seals by 0.25 mm and improving plate alignment.

Beyond the development of the activation mechanism, a remaining major subtask consists of in vitro testing of the prototype design against device performance and blood damage metrics, utilizing both hydraulic and hemolytic tests. Once completed, the resultant design was proven to provide effective transition between device operating modes with minimal impact on performance.

The design will incorporate improvements to the device interfaces and seals, to mitigate the potential for leaks to develop, as well as a separatable “catch can” below the activation mechanism seal to isolate and collect any potential leaks in the activation mechanism chamber.

The prototype was first evaluated using hydraulic testing, for leak testing and flow restriction evaluation. Experimental setup will be identical to the previous layout shown in FIG. 11B, utilizing the BioMedicus ⅜″ FloPump to provide the necessary pressure generation. The system will be run at 7 L/min of flow and a pressure differential of 150 mmHg, over at least 1 hour of support. To be deemed successful, the device must meet the specified pressure and flow requirements over the full duration of the test without leakage.

In addition, evaluation with hemolytic testing is performed based on protocols established in ASTM F1841-19e1, Standard Practice for Assessment of Hemolysis in Continuous Flow Blood Pumps. The tests utilize bovine blood. Specialized tubing (Terumo Cardiovascular, Tokyo, Japan), coated on blood contacting surfaces with Xcoating (a biopassive surface designed to mitigate confounding factors that could cause increased blood reactivity) are used throughout the entire circuit. Additionally, the Biomedicus ⅜″ FloPump is used to drive flow and pressure generation. Testing of the flow loop without the activation mechanism is performed first, to provide a control for the activation mechanism tests and to verify the flow loop independently against previous baseline data.

The flow loop setup is similar to FIG. 11B, with the addition of a water bath to heat the blood and a Terumo blood reservoir with Xcoating to hold excess blood and allow for sample collection. Tubing is run through a water bath heated to 37° Celsius and blood temperature is continually monitored using an IR temperature sensor. A sampling port at the outflow of the fluid reservoir allows for collection of blood throughout the test. Data collection is conducted over a period of 6 hours, where samples are collected in 1-hour increments and the normalized index of hemolysis (NIH) is calculated at each data point using the Cripps method, which calculates the plasma-free hemoglobin (pfHb) for each sample time point by reading the absorbance of the blood samples at three chosen wavelengths (terms A1, A2, and A3 in Equation 1). From there, NIH would be calculated from pfHb using Equation 2.

pfHb=(177.6*A1)−((A2+A3)/2) (Equation 1)

NIH=V(pfHb/tQ)((100−Hct)/100) (Equation 2)

In order to effectively analyze blood study data, a mean value for each time point is determined in each test set. Then, an ANOVA study will be performed to determine if each hourly mean is unique for each time point. Using a Pearson correlation test, an analysis compares NIH values to amount of time passed when the sample was taken, and NIH values are plotted against time to demonstrate the trend of NIH values as the study progresses. A regression analysis calculating the percent variation of time accounts for in NIH values was accomplished. A best fit equation is fit to the data set and evaluated for significance. When comparing multiple tests, a homogeneity of variance test was completed, using the Tukey-Hoaglin outlier test. The goal of this statistical analysis is to confirm that the data follows the expected distribution, and the experimental data is consistent across trials.

The final critical aspect in the development of the activation mechanism is to develop an accurate, consistent, and reliable method of actuation. Such an actuator should facilitate operational transition from axial-only operation to combined, tandem pump operation. Several actuator concepts were proposed in previous work, incorporating potential elements such as magnetic alignment, a lock-and-key mechanism, and a gear drive. These concepts will be thoroughly evaluated to generate a design which meets design requirements while fitting within the device housing; then the actuator will be tested on the benchtop to ensure repeatable operation (approximately 100 cycles without failure), failsafe operation (device will still provide flow in the event of cessation of mechanism power), and performance in line with design targets and goals.

Minor geometric adjustments may be required after these tests, and the final design prototype will incorporate the activation mechanism into the full design of the pump, and allow for testing of the full device, activation mechanism, and flow paths in tandem.

The primary outcome of this aim will be to develop an activation mechanism design which allows for safe and effective transition between device operating modes while meeting target motility, sealing, and blood damage requirements.

A further objective is to experimentally demonstrate the ability of the full device design to provide adequate support to pediatric patients, validating computational results through in vitro testing and quantitative examination of the device against the critical design requirements of size, pressure-capacity generation, and activation mechanism performance goals of leak-free operation and transition from single-pump to dual-pump support.

Both the pump flow paths and activation mechanism will be independently tested and verified. In vitro testing of an integrated prototype against outlined design requirements is the next step in the development of this device.

Shaft-drive prototypes of both the axial and centrifugal pumps in isolation, and experimental results demonstrated a close correlation with k-epsilon computational models. No in vitro studies incorporated additional portions of the full device flow path beyond the pumps; the initial design of the flow path was not evaluated computationally and exhibited several design limitations which would severely impact device performance, so such testing was not done. With the flow path and activation mechanism designs completed, in vitro testing of the fully integrated device allows for direct comparison of the holistic design against computational results and design requirements.

Initial experimental tests were isolated to the individual pump geometries, detached from the overall device flow path. This was done to verify the flow loop and check for consistency against previous work. Results from these experiments would be compared to both isolated pump simulations and previous in vitro test data. These tests utilized shaft drive to eliminate potential complications introduced by incorporating magnetic suspension.

The setup of the test loop was identical for all in vitro hydraulic testing; a diagram of the test loop can be seen in FIG. 13. The flow circuit consists of the pump housing and flow paths 152 supported on a mount 156, two custom fluid reservoirs, and a flow restriction clamp used to generate a pressure rise. The circuit was filled up with a 60% water-glycerol mixture, which more accurately matches the fluid dynamics of blood compared to pure water, and the circuit was bled to remove any bubbles. A Faulhaber 4490H204B electric motor (Faulhaber Drive Systems, Schonaich, Germany) 140 drove the pump(s) either directly or through a drive belt gear reduction, utilizing a drive shaft to spin the pump. The flow restriction clamp 142 was placed between the two fluid reservoirs, 144 and 146, and tightened to induce a differential pressure rise. This pressure was recorded as an electrical impulse using a differential pressure transducer (Validyne Engineering, Northridge, CA) 148, while flow rate was captured by an optical flow probe (Transonic Systems Inc, Ithaca, NY) 150 specifically calibrated for the water-glycerol solution. Data was collected using a Labjack and LJ Stream 154 for each operating condition, and each experiment was run in triplicate and time-averaged to determine the pressure rise and flow capacity performance. The axial pump was tested at speeds from 10,000 to 13,000 RPM and flow rates from 1-3 L/min, while the centrifugal pump was tested at speeds from 5,000-6,000 RPM and flow rates from 3-5 L/min. The resistance clamp was used to achieve the full flow range in 0.2 L/min increments.

The use of shaft drive required several small changes to the device and flow path geometries, in order to incorporate the drive shafts into the test rig. A sheath was added into the designs to protect the drive shaft and prevent the introduction of additional flow turbulence from an open rotating shaft. A shaft seal was installed outside of the main flow path in order to prevent leakage. Pump housing geometries beyond the inlet remained identical to match simulation setup as closely as possible. These initial benchmark tests verified the setup of the in vitro test rig; no issues arose with the test rig or data collection. Both the axial and the centrifugal pump test results correlated well with previous experimental test results.

The axial and combined device flow paths were incorporated into the experimental test rig. Utilizing the same test loop as the previous experiment, three tests were run: the axial flow path alone, the centrifugal flow path without the axial pump, and the combined flow path running both pumps in tandem. For the combined test, each pump was connected to its own motor and controlled independently. The following pump speeds were tested, at flow rates of 3-5 L/min: 5,000 (centrifugal)/11,000 (axial) RPM; 5,500/12,000 RPM; and 6,000/13,000 RPM. This allows for direct comparison with pump speeds evaluated in simulation.

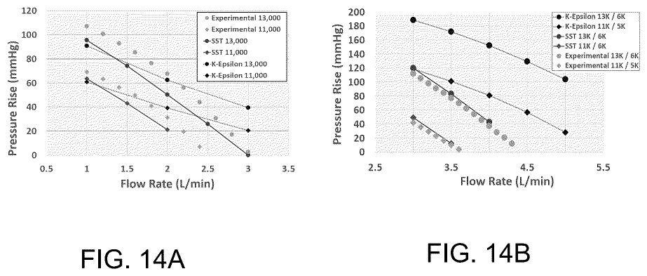

The results of the full flow path tests are shown in FIGS. 14A and 14B which provide a comparison of experimental results against simulation (k-epsilon and SST) results at multiple pump speeds for the axial-only flow path (FIG. 14A) and the combined, double-pump flow path (FIG. 14B). Both the axial tests and the combined tests demonstrated good correlation with SST simulation results; the axial test rig outperformed SST results by a small margin (maximum 15 mmHg) while the combined test rig slightly underperformed SST (maximum 8 mmHg). This overall delta was consistent across the operating range of the pump, at all rotational speeds. However, like the SST simulation predictions, the experimental data underperformed k-epsilon simulation results, most dramatically at higher flow rates.

In summation, there remains an unmet clinical need to innovate a pediatric MCS device which would be able to span the pediatric age range from infancy through adolescence and provide a long-term, single-device solution to clinical care providers. The embodiments disclosed herein provide innovative solutions that significantly advance the state of the art for pediatric MCS devices.

While the principles of the invention have been described above in connection with specific devices, systems, and/or methods, it is to be clearly understood that this description is made only by way of example and not as limitation. For instance, while a blood pump is described above, the disclosed device may be used in any mechanical circulatory support system. In addition, the pump design may also be used for other medical and non-medical purposes.

One of ordinary skill in the art will appreciate that various modifications and changes can be made without departing from the scope of the claims below. Accordingly, the specification and figures are to be regarded in an illustrative rather than a restrictive sense, and all such modifications are intended to be included within the scope of the present invention.

1. A mechanical circulatory support device, comprising:
a housing containing separate first and second pumps, each pump having an inlet, an outlet, and an impeller; and
an actuation mechanism within said housing and movable from a first position to a second position to divert blood flow within said housing to one of said pumps or to bypass blood flow relative to one of the pumps within the housing;
wherein, in the first position, the actuation mechanism directs blood flow exiting the first pump into a bypass passage so that blood flow bypasses the second pump, and wherein, in the second position, the actuation mechanism directs blood flow exiting the first pump into the inlet of the second pump;
wherein the first pump is an axial flow pump and the second pump is a centrifugal pump; and
wherein the actuation mechanism includes a movable flexible hose.

2. The device according to claim 1, wherein the actuation mechanism includes proximal and distal ends to which the flexible hose is connected and between which the flexible hose extends.

3. The device according to claim 2, wherein the proximal end is connected to the outlet of the axial pump.

4. The device according to claim 3, wherein the distal end includes an end plate to which the flexible hose is connected.

5. The device according to claim 4, wherein the device is movable to align the flexible hose to a flow path to the inlet of the centrifugal pump or to a bypass flow path.

6. The device according to claim 2, wherein the actuation mechanism is curved between the proximal and distal ends.

7. The device according to claim 1, wherein the actuation mechanism includes a slidable plate connected to a flexible hose extending from the outlet of the axial pump.

8. The device according to claim 7, wherein the plate is caused to slide between front and rear walls of the actuation mechanism.

9. The device according to claim 8, wherein the front wall of the actuation mechanism includes an elongate slot through which a connection of the flexible hose to the plate extends.

10. The device according to claim 9, wherein a linear actuator is connected to the plate and slides the plate relative to the front and rear walls of the actuation mechanism.

11. The device according to claim 10, wherein the rear wall defines separate ports for flow paths to the inlet of the centrifugal pump and to a bypass flow path.

12. A mechanical circulatory support device, comprising:
a housing containing separate first and second pumps, each pump having an inlet, an outlet, and an impeller; and
an actuation mechanism within said housing and movable from a first position to a second position to divert blood flow within said housing to one of said pumps or to bypass blood flow relative to one of the pumps within the housing;
wherein, in the first position, the actuation mechanism directs blood flow exiting the first pump into a bypass passage so that blood flow bypasses the second pump, and wherein, in the second position, the switching mechanism directs blood flow exiting the first pump into the second pump;
wherein the first pump is an axial flow pump and the second pump is a centrifugal pump; and
wherein the actuation mechanism is integrated within an outer wall of the centrifugal pump.

13. The device according to claim 8, wherein the actuation mechanism defines a blood flow diverting passage that is align-able with the inlet of centrifugal pump and a separate bypass passage that is align-able with an outlet of the device.
